(19) United States
(12) Patent Application Publication
(10) Pub. No.: US 2014/0091945 A1
(43) Pub. Date: Apr. 3, 2014

(54) SYSTEM AND METHOD FOR ATTACHMENT FREE MOTION, RESPIRATION, HEARTBEAT, AND VIDEO MONITORING

(71) Applicants: Edward Rivas, San Antonio, TX (US); Eric Neleigh, San Antonio, TX (US); Arturo Alejo Ayon, San Antonio, TX (US)

(72) Inventors: Edward Rivas, San Antonio, TX (US); Eric Neleigh, San Antonio, TX (US); Arturo Alejo Ayon, San Antonio, TX (US)

(21) Appl. No.: 13/890,487

(22) Filed: May 9, 2013

Related U.S. Application Data
(63) Continuation-in-part of application No. 12/576,230, filed on Oct. 8, 2009, now Pat. No. 8,502,679.
(60) Provisional application No. 61/644,688, filed on May 9, 2012.

(51) Int. Cl. G08B 21/02; A61B 5/00

(52) U.S. Cl. CPC G08B 21/0202; A61B 5/0024
USPC 340/870.01

(57) ABSTRACT

A system and method directed to attachment free monitoring of a subject's respiration, heartbeat, and motion. In one embodiment, the monitoring system is comprised of a monitoring pad, a base station, and a monitoring display device such as a smart phone or web interface. In another embodiment, the components of the system are communicatively coupled through wireless means. In yet another embodiment, the monitoring system additionally comprises a visual monitoring device. The monitoring pad provides the signal that is filtered to isolate various components representing the subject's motion, respiration, and heartbeat. The system may be programmed to provide alerts and subject stimulation based on various trigger points tied to thresholds in the measurements.

### CROSS-REFERENCE TO RELATED APPLICATIONS

This application claims the benefit under Title 35 United States Code §120 as a continuation-in-part application of U.S. patent application Ser. No. 12/576,230; Filed: Oct. 8, 2009, the full disclosure of which is incorporated herein by reference. This application also claims the benefit under Title 35 United States Code §119(e) of U.S. Provisional Patent Application Serial No. 61/644,688; Filed: May 9, 2012, the full disclosure of which is incorporated herein by reference.

### STATEMENT REGARDING FEDERALLY SPONSORED RESEARCH OR DEVELOPMENT

Not applicable

### THE NAMES OF THE PARTIES TO A JOINT RESEARCH AGREEMENT

Not applicable

### INCORPORATING-BY-REFERENCE OF MATERIAL SUBMITTED ON A COMPACT DISC

Not applicable

### SEQUENCE LISTING

Not applicable

### FIELD OF THE INVENTION

The present invention relates to a system and method directed to attachment free respiration, heartbeat, motion, and video monitoring of a subject. More specifically, the present invention relates to a system and method for attachment free respiration, heartbeat, motion, and video monitoring of a subject through a sensor pad communicatively coupled to a base station and the base station communicatively coupled to a remote monitoring device.

### BACKGROUND OF THE INVENTION

Without limiting the scope of the disclosed system and method, the background is described in connection with a novel system and approach to efficiently and effectively attachment free monitor a subject's motion, respiration, and heartbeat. The applications of this invention are directed to various environments such as medical facilities, home health, elderly care, and infant care. For example, in infant care it is desirable to monitor the sleeping activity of babies between the ages of birth and eighteen months due to the possibility of Sudden Infant Death Syndrome (S.I.D.S.). Unfortunately, there are about two thousand five hundred deaths per year in the United States, and thousands more worldwide. Of the major countries throughout the world, there are almost six thousand reported cases of S.I.D.S. annually. The various causes of S.I.D.S. are not known leaving two primary options to address the risk. The first option, which is passive, is to adhere to the recommendations from the major pediatric physician's group on safe sleeping including having the infant sleep on their back, removing all possible suffocation hazards from the sleeping space, and to have the infant sleep without blankets. This approach may indeed reduce the likelihood of S.I.D.S. but as a mere preventative measure, it still leaves the parent wondering about the status of their infant at any given time. A second option is to employ active monitoring of the infant.

The field's prior art reflects many approaches and devices in monitoring a subject's motion, respiration, and heartbeat. Many of these prior art references utilize invasive means to obtain the same results as the claimed invention.

A first example of a monitoring system in the prior art is described in U.S. Pat. No. 7,666,151 issued on Feb. 23, 2010 to Patrick K. Sullivan et al. In this example, the monitoring system also utilizes a different process to extract the components of the signal related to motion, respiration, and breathing. There is no subject stimulation employed by the system and the system architecture is still invasive from an implementation standpoint.

A second example of a monitoring system is described in U.S. Pat. No. 6,415,033 issued on Jul. 2, 2002 to Michael E. Haleck et al. This monitoring system utilizes the analysis of acoustic signals obtained from microphones. In addition, special cavities are employed to be able to take measurements for the analysis portion of the system

In reality, a monitoring system that utilizes external cables and wiring in its sensor implementation poses additional hazards and is still invasive to the monitoring environment. The current state of the prior art limits the effectiveness of a system in its implementation and accuracy in taking measurements. As a result, the monitoring is not as reliable or effective and is difficult to use.

While all of the aforementioned systems may fulfill their unique purposes, none of them fulfill the need for a practical, effective, and efficient means for attachment free monitoring of a subject's motion, respiration, and heartbeat.

Therefore, the present invention proposes a novel system and method for attachment free monitoring of a subject's motion, respiration, and heartbeat.

### BRIEF SUMMARY OF THE INVENTION

The present invention, therefore, provides a system and method directed to attachment free monitoring of a subject.

In one embodiment, the attachment free monitoring system obtains a subject's respiration, heartbeat, and motion through filtering of signals obtained from piezo benders mounted to a monitoring pad containing isolation dampeners and a layer of material for force distribution. The monitoring pad provides the signal that is then filtered to isolate various components representing the subject's motion, respiration, and heartbeat. In one embodiment, the monitoring system is comprised of a monitoring pad, a base station, and a monitoring display device such as a smart phone or web interface. In another embodiment, the components of the system are communicatively coupled through wireless means. In yet another embodiment, the monitoring system additionally contains a visual monitoring device. The system may be programmed to provide alerts and subject stimulation based on various trigger points tied to thresholds in the measurements.

In summary, the present invention discloses a system and method directed to attachment free monitoring of a subject's respiration, heartbeat, and motion. More specifically, the present invention relates to a system and method for monitoring of a subject's respiration, heartbeat, and motion through a sensor pad communicatively coupled to a base station and the base station communicatively coupled to a remote monitoring device.

### BRIEF DESCRIPTION OF THE SEVERAL VIEWS OF THE DRAWINGS

For a more complete understanding of the features and advantages of the present invention, reference is now made to the detailed description of the invention along with the accompanying figures in which:

FIG. 1 is a component diagram of the monitoring system in accordance with embodiments of the disclosure;

FIG. 2 is a top view of the piezo benders mounting configuration on isolation dampening material in accordance with embodiments of the disclosure;

FIG. 3 is a side view of the piezo benders mounting configuration on isolation dampening material in accordance with embodiments of the disclosure;

FIG. 4 is a side view of the monitoring pad comprising a rigid base, sensor grid, and a layer of foam in accordance with embodiments of the disclosure;

FIG. 5 is a schematic layout of the monitoring pad illustrating the various components in accordance with embodiments of the disclosure;

FIG. 6 is an exploded view of the monitoring pad illustrating the various components in accordance with embodiments of the disclosure;

FIG. 7 is a schematic layout of the base station front illustrating the various components in accordance with embodiments of the disclosure;

FIG. 8 is a schematic layout of the base station back illustrating the various components in accordance with embodiments of the disclosure;

FIG. 9 is a block flow diagram for the process utilized in the base station in accordance with embodiments of the disclosure;

FIG. 10 is a block flow diagram for the Fast Fourier Transform process utilized in the base station for the heart beat rate calculations in accordance with embodiments of the disclosure;

FIG. 11 is a block flow diagram for the auto-correlation process utilized in the base station for the heart beat rate calculations in accordance with embodiments of the disclosure;

FIG. 12 is a block flow diagram for the Fast Fourier Transform process utilized in the base station for the respiration rate calculations in accordance with embodiments of the disclosure;

FIG. 13 is a block flow diagram for the auto-correlation process utilized in the base station for the respiration rate calculations in accordance with embodiments of the disclosure.

FIG. 14 is a signal frequency versus magnitude representation to determine the subject's active or inactive status in accordance with embodiments of the disclosure.

### DETAILED DESCRIPTION OF THE INVENTION

Disclosed herein is an improved system and method for attachment free monitoring of a subject's motion, respiration, and heartbeat. In addition, video monitoring is incorporated as part of the device and method. The numerous innovative teachings of the present invention will be described with particular reference to several embodiments (by way of example, and not of limitation).

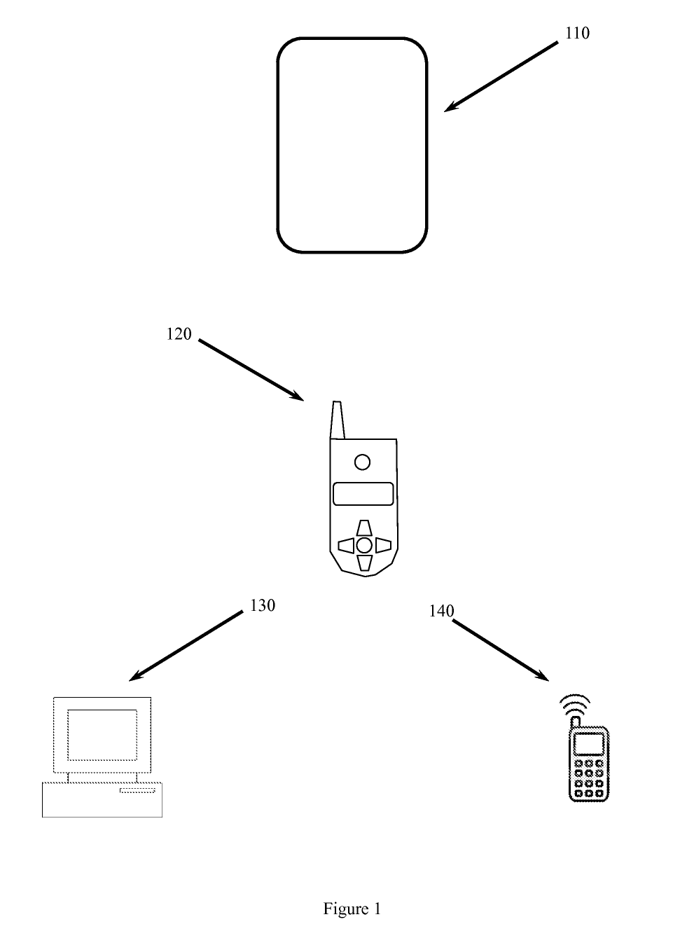

Reference is first made to FIG. 1, a component diagram of the monitoring system. The invention is an attachment free motion, respiration, heartbeat and video monitoring system for humans and animals. The system is comprised of a monitoring pad 110, base station 120, and monitoring devices such as a personal computer 130 or mobile device 140. The monitoring pad 110 contains no external wires or straps that may pose a hazard to the subject being monitored. For example, external wires and straps could cause the subject being monitored to become entangled or could be objects the subject being monitored could choke on. The subject lies upon the monitoring pad 110, which detects the motion of the subject through piezo benders integrated into the monitoring pad 110. Examples of motions picked up by the monitoring pad 110 include but are not limited to physical movements (such as the movements of limbs and falling out of the bed), respiration, and heartbeat. These motions are captured and transmitted wirelessly to the base station 120. The base station 120 contains the means for receiving and storing data as well as performing calculations on that data. The base station 120 takes the data captured and isolates the respiration and heartbeat data and then transmits the results to the monitoring devices 130, 140. Transmission of this data may take place through wireless or non-wireless means. The base station 120 is also able to send an alarm (visual, audible, physical) to the monitoring devices 130, 140 based on preset thresholds or if the energy of the signal drops to a preset level. The monitoring devices 130, 149 may be web enabled and/ or contain software applications as a graphical user interface (GUI) for the presentation layer of the monitoring system. Wireless communications may be established by having the base station 120 connected to a wireless router as one means for this type of communication.

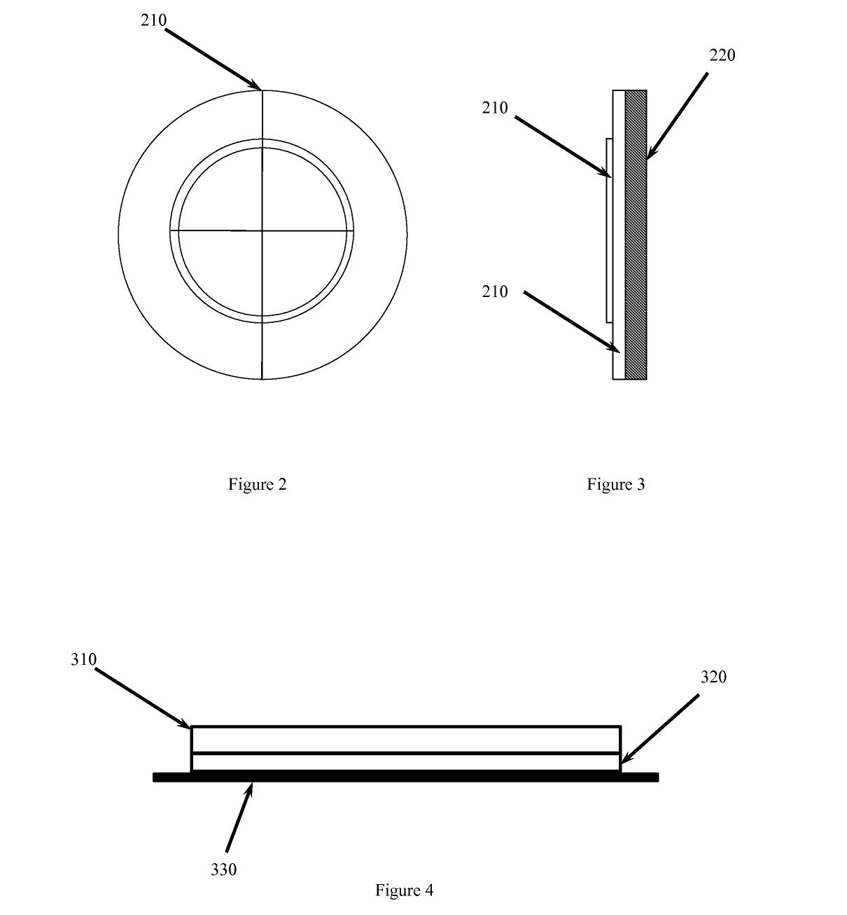

Reference is next made to FIGS. 2a and 2b, an illustration of the piezo benders 210 mounting configuration on isolation dampening material 220. FIG. 2a illustrates a top view of the piezo bender 210. In FIG. 2b, a side view of the mounting, the piezo benders 210 are mounted or coupled at the perimeter of the rings of isolation dampening material 220. That is, the isolation dampening material 220 is a ring with the center being open which allows the piezo benders 210 to be deflected more easily. In one embodiment, the dampening material is Sorbothane of durometer 40 and supports the perimeter of a round piezo bender 210.

Reference is next made to FIG. 3, a side view of a monitoring pad comprising a rigid base 330, an array of piezo benders also termed a sensor grid 320, and a layer of material such as foam 310. In one embodiment, the monitoring pad 110 consists of an array of piezo benders 320 mounted to the rigid base 330 by way of isolation dampeners, a layer of material 310 over the array of piezo benders 320 for force distribution and the subject's comfort, electronics to convert the piezo bender signals to digital form, and means to transmit the digital signals to the base station 120. The monitoring pad 110 would also contain the means to provide physical stimulation to the subject such as vibration and motions to awaken the subject. In another embodiment the layer of foam 310 is the isolation dampening material Sorbothane. The layer of foam 310 is placed over the sensor grid 320 to allow for better force distribution among the individual sensors in the sensor grid 320. Ideally, this layer of foam 310 would have a one-inch thickness with a firmness rating of 40. The monitoring pad 110 can be configured or manufactured to take on various shapes and sizes to adequately cover the area to be monitored.

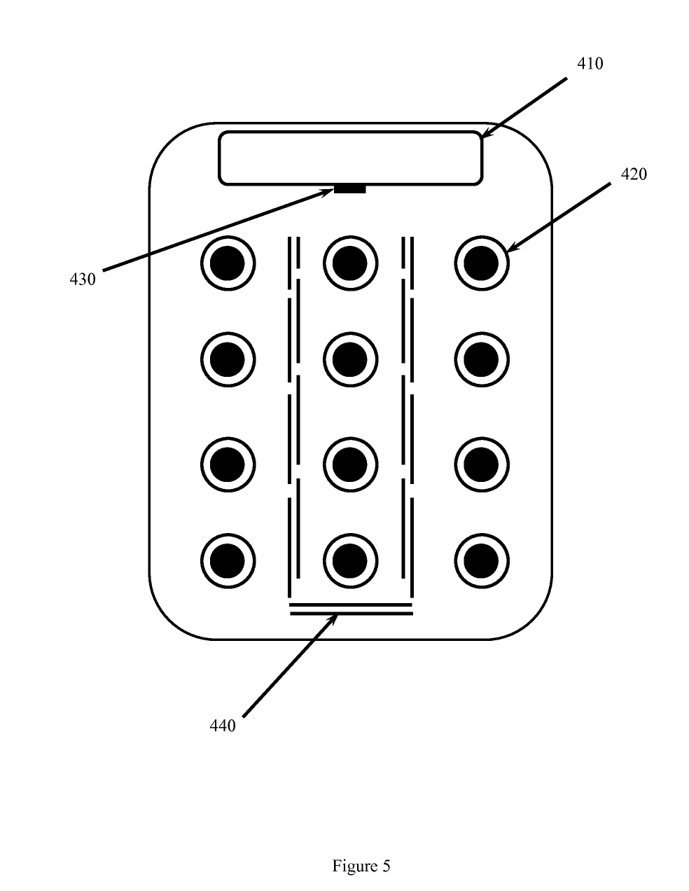

Reference is next made to FIGS. 4a and 4b, a schematic layout of the monitoring pad illustrating the various components and layout for design purposes. In FIG. 4a the electronics compartment 410, piezo benders 420, electronic box connector 430, and the wiring channels 440 can bee seen. In this embodiment the rigid base 330 has dimensions of fifteen inches by twenty-seven inches to fit in a standard baby bassinette. The sensor grid 320 has a layout of twelve piezo benders 420 in a four by three arrangement being five inches apart from center to center in the four piezo bender 420 direction and three point seven five inches apart in the three piezo bender 420 direction wired in series to an A/D input. Electrically, six piezo benders 420 are wired in series from ground to a center biased 1.1 V source and six peizo benders 420 are wired in series from the same point to 2.2 V, which is the maximum input range of the A/D used. A microprocessor controls the timing of the acquisition as well as the transmission of collected data to the base station 120. An example of a processor used for data acquisition of the signal generated by the PUI piezo bender array is the Microchip PIC18F25K80. Force applied to any individual piezo bender 420 will be additive to the output of all other piezo benders 420 from the point of view of the A/D input. The analog input can also be amplified through one of four amplifications (1×, 2×, 4×, 8×) before the A/D conversion process. This amplification is software selectable. Data transmission to the base station 120 is accomplished by way of ANT Wireless Personal Network protocol. Power is supplied by two AA batteries which are contained with all the digital electronics in a compartment 410 isolated form the piezo array. While this is illustrative of one embodiment, other embodiments may employ alternative power sources such as capacitors, solar, wireless transmission, motion of the subject, or even thermal energy from the subject being monitored. A fifteen hertz sampling rate provides good resolution of biological indicators of interest while allowing for low power consumption for acquisition and transmission of data. In other embodiments the rigid base is soft and/or flexible as illustrated in FIG. 4b. An example of a soft base is a base made of the material EVA foam. FIG. 4b is a perspective view of the monitoring pad illustrating the electronics compartment 410, the piezo benders 420, the soft base 430, and the foam layer 440. An advantage of having a soft base is allowing the piezo benders to deflect freely without any rigid support.

Reference is next made to FIG. 5, a schematic layout of the base station 120 illustrating the various components. The base station 120 contains the means such as electronic to receive data, process data to isolate the components for respiration and heartbeat, store data, transmit current or stored data to another device such as the monitoring devices 130, 140, determine of an alert should be triggered, and transmit the alert status to another device such as the monitoring devices 130, 140. In one embodiment, the base station 120 is comprised of a microprocessor (within the base station), local display 530, camera 520, microphone 590, wi-fi antenna 510, network connection 560, external power connection 580, control pad 550, indicator lights 540, 555, and mounting point 570. An example of an implementation is a Freescale IMX53 microprocessor based system with the capability to stream video, audio, signal data, processed data, and calculated data to monitoring devices 130, 140 or any other devices utilized in the monitoring system. The processor can be running Ubuntu linux. The camera 520 which allows for video of the subject to be taken and broadcast to another device. The camera 520 has an infrared illumination assist consisting of a ring of infrared LED which provide illumination of the subject with light detectable by the camera 520, but will not disturb the subject. The local display 530 and control pad 550 allows for basic controls and status displays of the system locally. The wi-fi antenna 510 and network connection 560 allows for communications of the base station 120 with other devices such as the remote devices 130, 140. The indicator lights 540, 555 allow for easy visual status identification when the local display 530 is not viewable. The mounting point 570 allows for the base station 120 to be mounted easily to items such as a wall bracket or stand. Data collected and calculations performed can be stored in long-term memory in the form of a searchable database. An external device, connected by way of a wireless router, can perform the searches. The microphone 590, is utilized to obtain audio feedback of the monitoring environment. The external power connection 580 is utilized to connect the base station to an external power source.

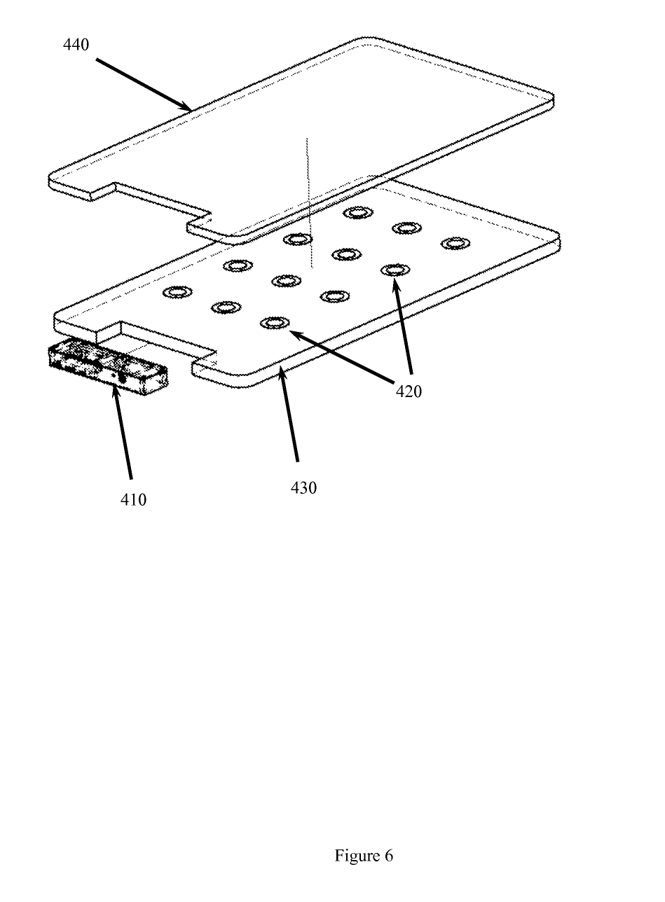

Reference is next made to FIG. 6, a block flow diagram for the process utilized in the base station 120. The DC component or average is subtracted from the time based signal collected from the monitoring pad 110. This signal is passed through a 3^{rd} order Butterworth filter for extracting the signals of interest for both the respiration and heart beat information. For human infants, a band pass region of between 0.33 Hz and 1.33 Hz is utilized for detecting the respiration information such as breath rate. Also for human infants, a band pass region of between 1.33 Hz and 3.00 Hz is utilized for detecting the heart beat information such as rate. In other embodiments, a filter region can be chosen for a specific gender, age range, or species.

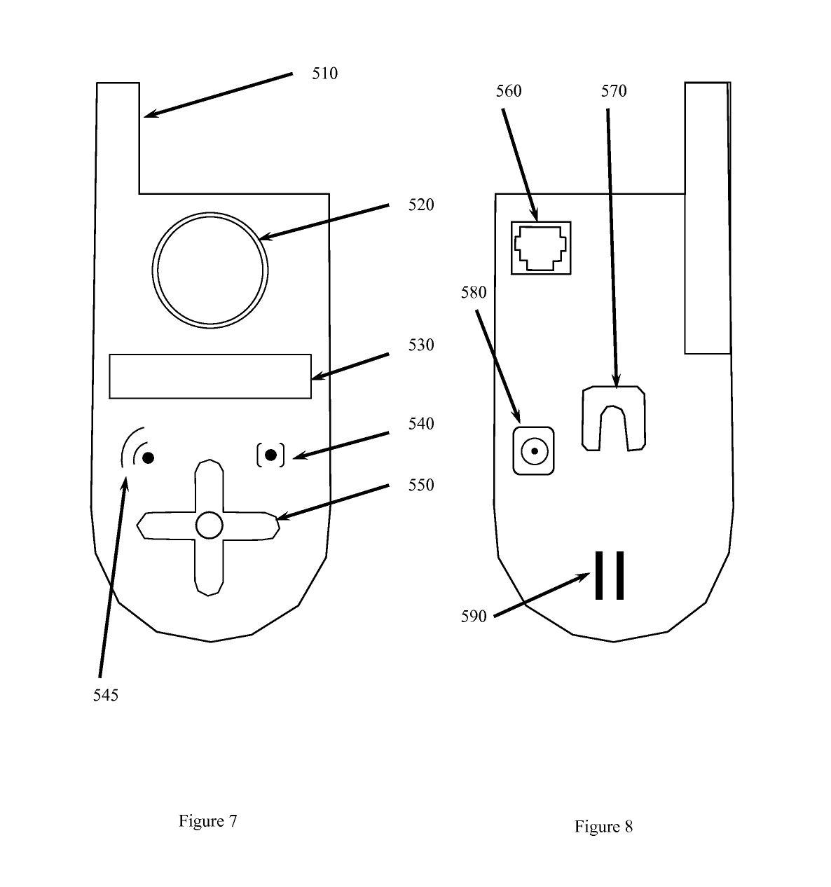

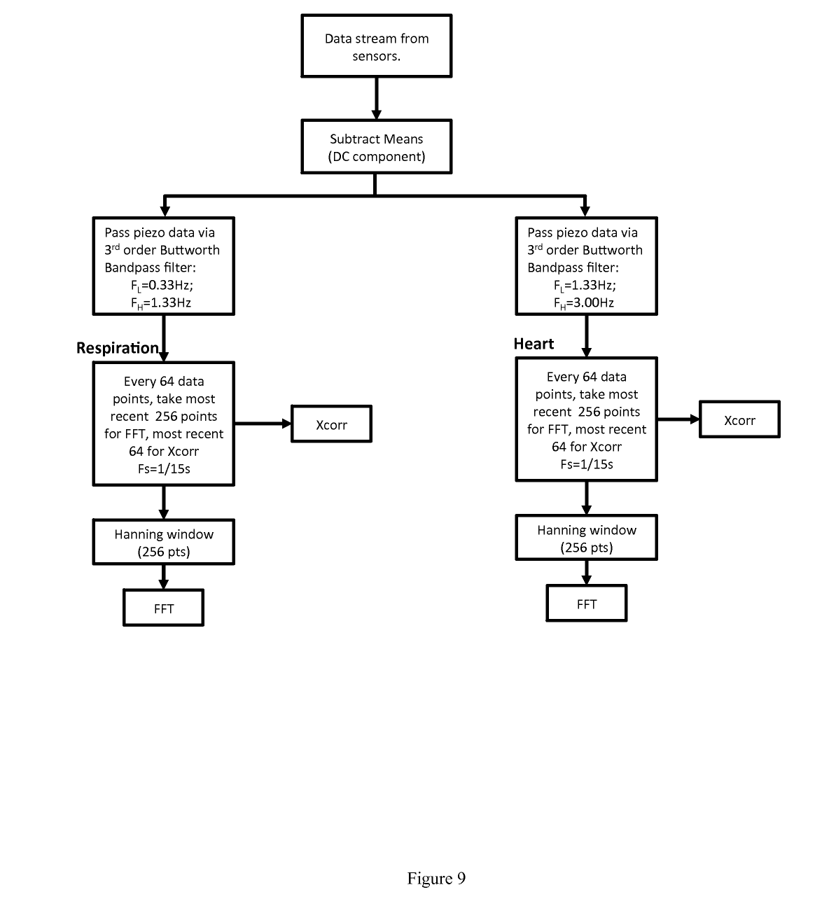

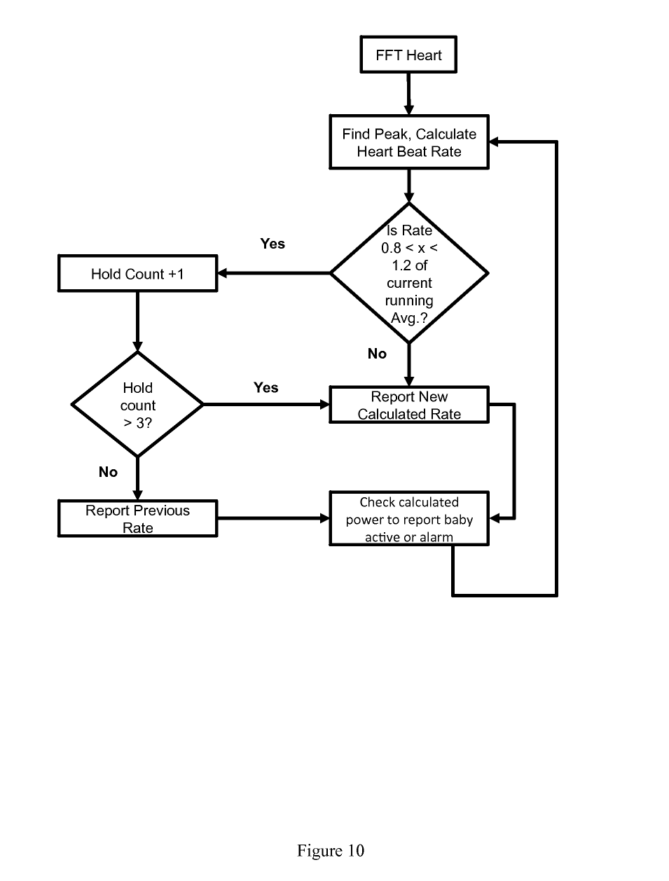

Reference will now be made to FIG. 7 and FIG. 9, block diagrams for the Fast Fourier Transform (FFT) process utilized in the base station 120 for the heart beat rate and respiration rate calculations. For respiration and heart beat information, a Fast Fourier Transform (FFT) is performed on a 256 sample set of the filtered respiration and heart beat signal respectively, which has been windowed by a 256 point Hanning filter. The frequency of the highest energy content is considered to be the corresponding respiration rate or heart beat rate for the latest calculation iteration. By finding peak and calculating respiration and heart beat rate, this is referencing the conversion of the peak from cycles per second to cycles per minute. Reference will next be made to FIG. 8 and FIG. 10, block diagrams for the auto correlation process utilized in the base station 120 for the heart beat rate and respiration rate calculations. Another aspect of the algorithm is the auto correlation of a 64 point sample set is used to determine the fundamental period of respiration rate or heart beat rate, which would allow for quicker calculation times. To reduce the chances of a spurious reading to trigger a false alarm, an immediate change of calculated rates may not trigger an alarm. Instead, a moving average window is utilized to determine if an immediate reading is likely to be accurate.

Reference will now be made to FIG. 8. For the case of respiration rate, a running average of calculated respiration rates from the last three calculations is kept. If the current calculation falls within a window of the current average multiplied by 0.9 and the current average multiplied by 1.1, it is deemed to be accurate. If it falls outside this window, the previous calculated respiration rate is held for an additional iteration. However, the new calculated value is used for the purposes of keeping the running average.

Reference is next made to FIG. 10. In the case of heart beat rate, a running average of calculated heart beat rates from the last three calculations is kept. If the current calculation falls within a window of the current average multiplied by 0.8 and the current average multiplied by 1.2, it is deemed to be accurate. If it falls outside this window, the previous calculated heart rate is held for an additional iteration. However, the new calculated value is used for the purposes of keeping the running average. In other embodiments, a different moving average range might be utilized or the depth of the moving average might be changed. In the exemplary embodiment of the system, a set of alarms can be issued depending on the respiration rate calculated or the heart beat rate calculated, as well as the overall energy detected in the biological indicators of interest. An overall energy level in the region of interest can indicate a problem with a shallowness of breathing or weak pulse or that the subject is moving or thrashing around. If the subject is moving around too much to get a clear respiration rate or heart beat rate, an active signal can be sent that there is motion on the monitoring pad 110, but no clear reading of biological indicators. If the overall energy is too low to be reasonable or to get an accurate reading of biological indicators, and alarm signal can be sent. In the case of detecting the overall energy in the region of interest, a smaller sample point set of 64 samples for the FFT can be utilized to trigger an alarm or active state faster than it would be capable while calculating the biological rates at a higher resolution.

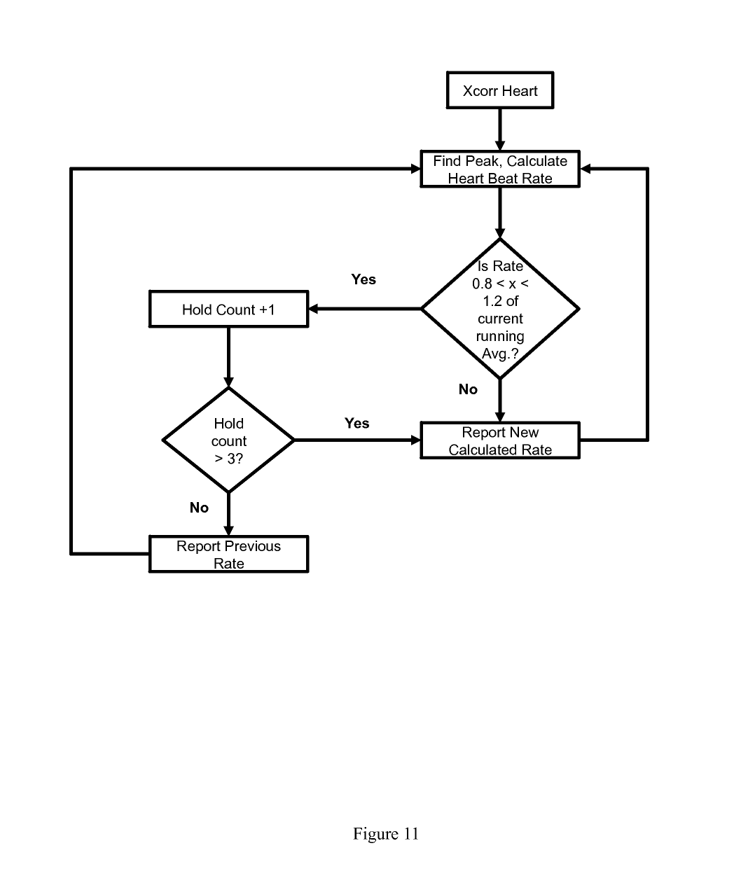

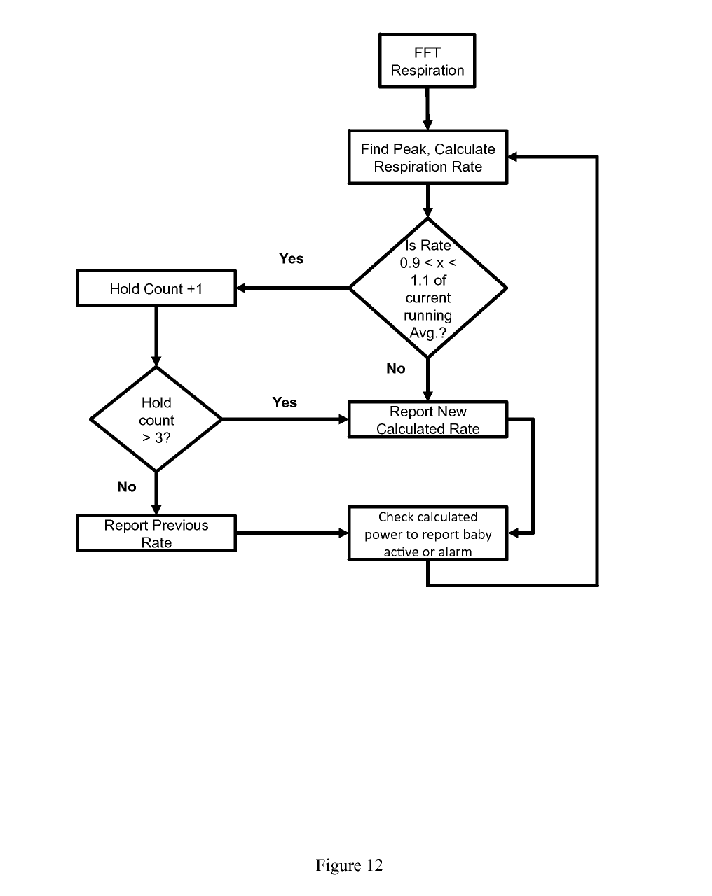

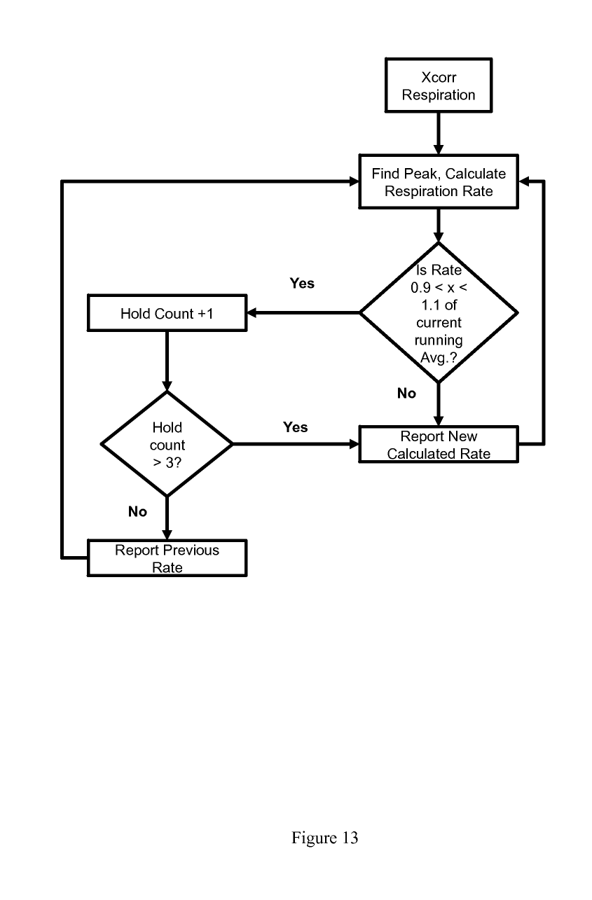

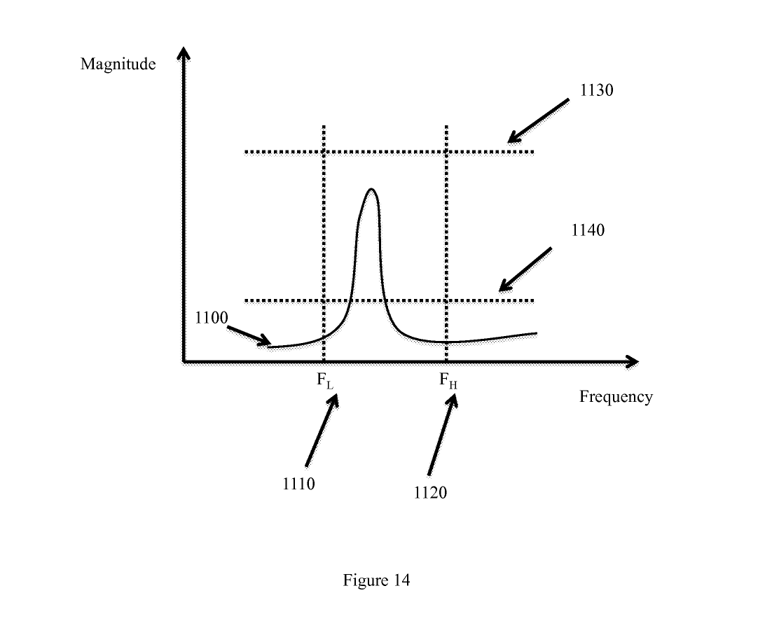

Reference is lastly made to FIG. 11, a signal frequency versus magnitude representation to determine the subject's active or inactive status. As previously mentioned, the subject lies upon the monitoring pad 110, which detects the motion of the subject through piezo benders integrated into the monitoring pad 110. Examples of motions picked up by the monitoring pad 110 include but are not limited to physical movements (such as the movements of limbs and falling out of the bed), respiration, and heartbeat. The differentiating criteria for determining whether the subject is moving, having normal respiration and heartbeat or is missing is done using a combination of the magnitude of the sampled signal as well as the frequency of the energy peaks in the frequency domain representation, represented in FIG. 11. In order to detect a respiratory rate or heartbeat rate, the circuitry is configured to amplify the sensor signal so that the dynamic range is matched to the input range of the analog to digital converter. In the event of an episode where the subject is moving, there is a high likelihood that the input amplifiers would be saturated, or producing a maximum output voltage. In this event, the system cannot calculate the vital signs but it can be inferred that if the signal magnitude 1100 exceeds a certain threshold 1130 then we can safely assume that the subject is alive but active. In the infant monitor implementation, this state is called “Baby Active”. If the signal magnitude falls below a certain minimum threshold 1140 then the subject is assumed to have very shallow breathing movements or is no longer on the sensor pad. 1110 and 1120 indicate the algorithm's low and high frequency thresholds for a frequency peak, outside of which an alarm is triggered.

In brief, the system and method is directed to attachment free monitoring as described herein and provides for an effective and efficient means for monitoring a subject's motion, respiration, and heartbeat. In addition, video monitoring is incorporated as part of the device and method.

The disclosed system and method is generally described, with examples incorporated as particular embodiments of the invention and to demonstrate the practice and advantages thereof. It is understood that the examples are given by way of illustration and are not intended to limit the specification or the claims in any manner.

To facilitate the understanding of this invention, a number of terms may be defined below. Terms defined herein have meanings as commonly understood by a person of ordinary skill in the areas relevant to the present invention. Terms such as “a”, “an”, and “the” are not intended to refer to only a singular entity, but include the general class of which a specific example may be used for illustration. The terminology herein is used to describe specific embodiments of the invention, but their usage does not delimit the disclosed system or method, except as may be outlined in the claims.

Alternative applications for this invention include using this system and method for obtaining video, motion, respiration, and heart beat in other machines and applications. Consequently, any embodiments comprising a one piece or multi piece system having the structures as herein disclosed with similar function shall fall into the coverage of claims of the present invention and shall lack the novelty and inventive step criteria.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific system and method of use described herein. Such equivalents are considered to be within the scope of this invention and are covered by the claims.

All publications and patent applications mentioned in the specification are indicative of the level of those skilled in the art to which this invention pertains. All publications and patent application are herein incorporated by reference to the same extent as if each individual publication or patent application was specifically and individually indicated to be incorporated by reference.

In the claims, all transitional phrases such as “comprising,” “including,” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

The system and/or methods disclosed and claimed herein can be made and executed without undue experimentation in light of the present disclosure. While the system and methods of this invention have been described in terms of preferred embodiments, it will be apparent to those skilled in the art that variations may be applied to the system and/or methods and in the steps or in the sequence of steps of the method described herein without departing from the concept, spirit, and scope of the invention.

More specifically, it will be apparent that certain components which are both shape and material related may be substituted for the components described herein while the same or similar results would be achieved. All such similar substitutes and modifications apparent to those skilled in the art are deemed to be within the spirit, scope, and concept of the invention as defined by the appended claims.

What is claimed is:

1. A non-invasive monitoring system comprising:
a pad for allowing a subject to lay on;
a sensor array comprising impulse-sensitive transducers, the sensor array configured to be integrated with said pad and to provide a time-domain composite output waveform responsive to said subject's motion, respiration, and heart beat;
a subject agitator;
a signal processor configured to:
receive the time-domain composite output waveform;
filter the time-domain composite waveform to obtain the time-domain respiration rate signal waveform and the heart rate signal waveform;
apply a Fast Fourier Transform to the time-domain respiration rate signal waveform and the heart rate signal waveform to provide frequency-domain signal waveforms of the respiration rate and the heart rate; and
find the peak of each corresponding signal to calculate the respiration rate and the heart beat rate.

2. The system of claim 1 wherein the sensor array comprises piezoelectric sensors.

3. The system of claim 1 wherein the sensor array comprises piezo benders sensors.

4. The system of claim 1 wherein the impulse sensitive transducers are connected in series.

5. The system of claim 1 wherein the output of the sensor array is biased to a desired output level by means of a voltage divider.

6. The system of claim 1 wherein:
the sensor array provides the time-domain output waveform to the signal processor by means of an analog-to-digital converter; and
the signal processor is a field-programmable gate array configured to receive a digital input signal.

7. The system of claim 1 wherein the subject agitator is a physical agitator configured to gently agitate the pad to awake the subject.

8. The system of claim 1 wherein the subject agitator is a shrill alarm configured to sit near the pad.

9. The system of claim 1 further comprising a wireless link to a monitoring unit, the monitoring unit configured to:
receive a signal from the alert state; and
provide a notification alarm.

10. The system of claim 1 wherein the signal processor is further configured to filter the time-domain composite signal through a first bandpass filter having a pass band between 0.33 and 1.33 hertz to provide a time-domain respiration signal, and filter the time-domain composite waveform through a second bandpass filter having a pass band between 1.33 and 3.00 hertz to provide a time-domain heart rate signal.

11. The system of claim 1 wherein the signal processor is further configured upon failing to detect either a frequency-domain respiration signal or a frequency-domain heart rate signal for a time period, enter an alert state and activate the subject agitator.

12. The system of claim 1 wherein the signal processor is further configured upon failing to detect either a frequency-domain respiration signal or a frequency-domain heart rate signal for a time period between two and twenty seconds, enter an alert state and activate the subject agitator.

13. The system of claim 1 further comprising a wireless link to a monitoring unit, the monitoring unit configured to:
receive motion, respiration rate, and heart beat rate data;
storage of said data;
provide said data for monitoring purposes; and
provide a notification alarm on set thresholds.

14. The system of claim 1 further comprising:
a video recorder, the video recorder configured to:
video record the subject while non-invasively being monitored;
and a wireless link to a monitoring unit, the monitoring unit configured to:
receive video, motion, respiration rate, and heart beat rate data;
storage of said data;
provide said data for monitoring purposes; and
provide a notification alarm on set thresholds.

15. The system of claim 1 wherein the sensor array comprises piezo bender sensors coupled with isolation dampening material along the perimeter of the ring.

16. A method of non-invasively monitoring motion, heart rate, and respiration rate of a subject comprising the steps of:
receiving a time-domain composite signal from a impulse-sensitive sensor array comprising impulse-sensitive transducers, the time-domain composite signal comprising a respiration rate component and a heart rate component;
determining if the signal indicates an active or inactive state;
filtering the time-domain composite waveform to obtain the time-domain respiration rate signal waveform and the heart rate signal waveform;
applying a Fast Fourier Transform function to the time-domain respiration rate signal waveform and the heart rate signal waveform to provide frequency-domain signal waveforms of the respiration rate and the heart beat rate; and
finding the peak of each corresponding signal to calculate the respiration rate and the heart beat rate.

17. The method of claim 16 further comprising the step of providing an alert if either the frequency-domain heart rate signal or the frequency-domain respiration rate signal is interrupted for a time span.

18. The method of claim 16 further comprising the step of providing an alert if either the frequency-domain heart rate signal or the frequency-domain respiration rate signal is interrupted for a time span between two and twenty seconds.

19. The method of claim 16 further comprising the step of providing a shrill alarm to the subject upon receiving the alert.

20. The method of claim 16 further comprising the step of automatically gently agitating the subject upon receiving the alert.
